(19) United States
(12) Patent Application Publication
(10) Pub. No.: US 2019/0269740 A1
(43) Pub. Date: Sep. 5, 2019

(54) USE OF PROBIOTIC BACTERIAL STRAINS AND CELL EXTRACTS TO INHIBIT ACIDOSIS AND LIVER ABSCESSES IN CATTLE

(71) Applicant: NUtech Ventures, Lincoln, NE (US)

(72) Inventor: Samodha Charaka Fernando, Lincoln, NE (US)

(21) Appl. No.: 16/344,968

(22) PCT Filed: Oct. 26, 2017
(86) PCT No.: PCT/US17/58562

§ 371 Date: Apr. 25, 2019

Related U.S. Application Data
(60) Provisional application No. 62/413,054, filed on Oct. 26, 2016.

(51) Int. Cl. A61K 35/741; A23K 10/18; A23K 20/163; A23K 50/10; A61K 9/19; A61K 47/10

(52) U.S. Cl. CPC A61K 35/741; A23K 10/18; A61K 47/10; A23K 50/10; A61K 9/19; A23K 20/163

(57) ABSTRACT

Disclosed are methods of using probiotic bacterial strains Aneurinibacillus migulanus, Aneurinibacillus aneurinilyticus and/or extracts thereof for inhibiting the growth of Streptococcus bovis, Fusobacterium necrophorum, and Acranobacterium (Actinomyces) pyogenes. These bacterial strains and/or their extracts can be used in cattle to treat acidosis and/or liver abscesses, as well as inhibit foot rot, among other infections and conditions caused by Streptococcus bovis and/or Fusobacterium necrophorum and/or Acranobacterium (Actinomyces) pyogenes. Compositions for use in these methods are also provided.

### INCORPORATION OF SEQUENCE LISTING

A paper copy of the Sequence Listing and a computer readable form of the Sequence Listing containing the file named “2016-079 ST25.txt”, which is 2,582 bytes in size (as measured in MICROSOFT WINDOWS® EXPLORER), are provided herein and are herein incorporated by reference. This Sequence Listing consists of SEQ ID NOs:1-2.

### BACKGROUND OF THE DISCLOSURE

The present disclosure relates generally to the use of bacterial strains useful as probiotics. In some forms, these bacterial strains synthesize and excrete antimicrobial compounds and extracts that have beneficial effects. More particularly, the present disclosure is directed to the use of probiotic bacterial strains Aneurinibacillus migulanus and Aneurinibacillus aneurinilyticus and their extracts for inhibiting the growth of Streptococcus bovis, Fusobacterium necrophorum and Acranobacterium (Actinomyces) pyogenes. These bacterial strains and their extracts can be used in cattle to treat acidosis and/or liver abscesses, as well as inhibit foot rot, and other infections and conditions caused by Streptococcus bovi and/or Fusobacterium necrophorum and/or Acranobacterium (Actinomyces) pyogenes.

Liver abscesses are seen in all ages and breeds of cattle. They are most common in feedlot and dairy cattle fed rations that are high in fermentable starches that predispose the animal to acidosis and rumenitis. Cattle with acidosis and liver abscesses have reduced production efficiency. The affected livers are condemned at slaughter, and adhesions to surrounding organs or the diaphragm may necessitate carcass trimming. Liver abscesses can also lead to disease syndromes associated with posterior vena caval thrombosis.

Fusobacterium necrophorum, a gram-negative, obligate anaerobic bacterium, and a component of normal rumen microflora, is the primary etiologic agent of liver abscesses. Infection in the liver usually originates from a necrobacillary rumenitis. Two biovars have been implicated. Biovar A (F. necrophorum necrophorum), the more virulent, is the predominant biovar in the rumen microflora and is isolated, usually in pure culture, from most cases of liver abscessation. Biovar B (F necrophorum funduliforme) is commonly isolated from microabscesses in the rumen wall, but is less commonly isolated from liver abscesses, in which it is always found in mixed culture with biovar A or other bacterial species. Acranobacterium (Actinomyces) pyogenes, Streptococci, Staphylococci, and Bacteroides spp are most frequently recovered from mixed cultures.

Rumenitis is usually the result of rapid intra-ruminal fermentation of dietary carbohydrate with subsequent production of lactic acid and increased acidity of the ruminal fluid (“grain overload”). Rations with high levels of carbohydrate are the principal cause in both dairy and feedlot cattle, but the texture of the feed and method of feeding can be modifying factors. The incidence of rumenitis in feedlot cattle is significantly higher when the cattle are transferred directly from a roughage ration to a high starch (high energy) finishing ration, and when there is poor feed bunk management. F necrophorum, alone or with other bacteria, colonizes through the area of superficial necrosis produced due to the acidity of the rumen contents. Leukotoxin may facilitate resistance to phagocytosis. Bacterial emboli from the lesions invade the hepatic portal venous system and are transported to the liver, where they can establish infectious foci of necrobacillosis that eventually develop into abscesses.

While there are current therapies available for controlling liver abscesses, these therapies have met with little success. For example, while tylosin phosphate has been found to reduce the number of liver abscesses and increases feed efficiency and weight gain, it has little, if any, effect on prevalence of ruminal lesions. Further, with dairy cattle, percutaneous drainage and long-term therapy with procaine penicillin G can be attempted, but the prognosis is poor. Accordingly, the primary control is by managing ruminal acidosis through the method of feeding, adaptation using diet composition, diligent feed bunk management, and use of buffers in the diet.

Based on the foregoing, there is a need in the art for alternative therapies for reducing acidosis and/or liver abscesses in livestocks, particularly, cattle. In some forms, it would be beneficial if the therapy could be added directly to feed as a feed additive, or as a direct fed microbial (DFM) product. In other forms, it would be beneficial if the therapy could be formulated for topical application.

### BRIEF DESCRIPTION OF THE DISCLOSURE

The present disclosure is generally related to the use of probiotic bacterial strains Aneurinibacillus migulanus and Aneurinibacillus aneurinilyticus and/or extracts thereof for inhibiting the growth of Streptococcus bovis, Fusobacterium necrophorum and Acranobacterium (Actinomyces) pyogenes. Growth of Streptococcus bovis, Fusobacterium necrophorum and Acranobacterium (Actinomyces) pyogenes has been linked to acidosis, which leads to liver abscesses in livestock, as well as foot rot, particularly in feedlot and dairy cattle. Accordingly, by inhibiting the growth, the present disclosure can provide treatment for these conditions and ailments.

Accordingly, in one aspect, the present disclosure is directed to a method of inhibiting Streptococcus bovis in a subject in need thereof, the method comprising administering to the subject an effective amount of a bacterial strain selected from the group consisting of Aneurinibacillus migulanus, Aneurinibacillus aneurinilyticus or an extract thereof and combinations thereof. Preferably the inhibition is at least 5%, more preferably at least 10%, still more preferably at least 20%, even more preferably at least 30%, still more preferably at least 40%, even more preferably at least 50%, still more preferably at least 60%, even more preferably at least 70%, still more preferably at least 80%, even more preferably at least 90%, and most preferably between 95% and 100%.

In another aspect, the present disclosure is directed to a method of inhibiting Fusobacterium necrophorum and Acranobacterium (Actinomyces) pyogenes in a subject in need thereof, the method comprising administering to the subject an effective amount of a bacterial strain selected from the group consisting of Aneurinibacillus migulanus, Aneurinibacillus aneurinilyticus or an extract thereof and combinations thereof. Preferably the inhibition is at least 5%, more preferably at least 10%, still more preferably at least 20%, even more preferably at least 30%, still more preferably at least 40%, even more preferably at least 50%, still more preferably at least 60%, even more preferably at least 70%, still more preferably at least 80%, even more preferably at least 90%, and most preferably between 95% and 100%.

In yet another aspect, the present disclosure is directed to a method of reducing the incidence or severity of acidosis in a subject in need thereof, the method comprising administering to the subject an effective amount of a bacterial strain selected from the group consisting of Aneurinibacillus migulanus, Aneurinibacillus aneurinilyticus or an extract thereof and combinations thereof. Preferably the reduction is at least 5%, more preferably at least 10%, still more preferably at least 20%, even more preferably at least 30%, still more preferably at least 40%, even more preferably at least 50%, still more preferably at least 60%, even more preferably at least 70%, still more preferably at least 80%, even more preferably at least 90%, and most preferably between 95% and 100%.

In yet another aspect, the present disclosure is directed to a method of reducing the incidence or severity of liver abscesses or foot rot in a subject in need thereof, the method comprising administering to the subject an effective amount of a bacterial strain selected from the group consisting of Aneurinibacillus migulanus, Aneurinibacillus aneurinilyticus or an extract thereof and combinations thereof. In some forms, the reduction in the incidence of liver abscesses is in comparison to a similar sized animal or herd of animals and is based on the total number of liver abscesses or foot rot lesions or the percentage of animals with liver abscesses or foot rot. Preferably the reduction is at least 5%, more preferably at least 10%, still more preferably at least 20%, even more preferably at least 30%, still more preferably at least 40%, even more preferably at least 50%, still more preferably at least 60%, even more preferably at least 70%, still more preferably at least 80%, even more preferably at least 90%, and most preferably between 95% and 100%.

In another aspect, the present disclosure is directed to a method of treating foot rot in a subject in need thereof, the method comprising administering to the subject an effective amount of a bacterial strain selected from the group consisting of Aneurinibacillus migulanus, Aneurinibacillus aneurinilyticus or extract thereof and combinations thereof.

In yet another aspect, the present disclosure is directed to a method of controlling liver abscesses and/or foot rot in an animal or herd of animals, the method comprising administering an effective amount of a bacterial strain selected from the group consisting of Aneurinibacillus migulanus, Aneurinibacillus aneurinilyticus or an extract thereof and any combination thereof to an animal or to the animals of the herd. In some forms, controlling liver abscesses and/or foot rot is in comparison to the animal or herd of animals and is based on the total number of liver abscesses or foot rot areas or the percentage of animals with liver abscesses or foot rot before and after receiving a composition comprising at least one of the bacterial strains. A further comparison can be made to a similar sized animal or herd of animals that has not received administration of a composition comprising at least one of the bacterial strains. In other forms, controlling is measured by comparing the severity of the liver abscesses or foot rot before and after receiving an administration of a composition comprising at least one of the bacterial strains. Preferably evidence of controlling is provided by having at least 5% less progression in severity or in the number of liver abscesses or areas of foot rot or their severity, more preferably at least 10%, still more preferably at least 20%, even more preferably at least 30%, still more preferably at least 40%, even more preferably at least 50%, still more preferably at least 60%, even more preferably at least 70%, still more preferably at least 80%, even more preferably at least 90%, and most preferably between 95% and 100% after receiving an administration of a composition comprising the bacterial strains. In some forms, this progression is measured by comparing the number of liver abscesses or areas of foot rot from before the administration of a composition comprising at least one of the bacterial strains to the number of liver abscesses or areas of foot rot after the administration of a composition comprising at least one of the bacterial strains.

In another aspect, the present disclosure is directed to a method of treating foot rot in a subject in need thereof, the method comprising administering to the subject an effective amount of a bacterial strain selected from the group consisting of Aneurinibacillus migulanus, Aneurinibacillus aneurinilyticus or extract thereof and any combination thereof. In some forms, the administration comprises the step of applying an amount of at least one of the bacterial strains to the feet of a subject in need thereof. In some forms, the bacterial strains will be incorporated into a gel, cream, spray, or lotion. In other forms, the bacterial strains will a component in an aqueous medium that will be applied topically to an area affected by foot rot. Preferred routes of topical administration include spraying, submersion in a bath, and the like.

In yet another aspect, the present disclosure is directed to a composition comprising an amount of at least one bacterial strain selected from the group consisting of Aneurinibacillus migulanus, Aneurinibacillus aneurinilyticus or extract thereof and any combination thereof. In some forms of the composition, both bacterial strains are present. In some forms of the composition, the bacterial strains include live cultures combined with feed. In some forms of the composition, the bacterial strains are lyophilized. Such lyophilized forms can be reconstituted or administered in their lyophilized form. Such lyophilized forms can also be combined with feed. In some forms of the composition including live cultures or lyophilized bacterial strains, the bacterial strains are combined or mixed with a prebiotic. Such combinations can be added to feed. In some forms of the composition, the composition is formulated for oral administration. In some forms of the composition, the composition is formulated for topical formulation.

In yet another aspect, the present disclosure is directed to a kit comprising the composition as described herein. In some forms, the kit includes a container for housing the components of the kit. In some forms, the kit includes instructions for use.

In still another aspect, the present disclosure is directed to a foot bath comprising an amount of a bacterial strain selected from the group consisting of Aneurinibacillus migulanus, Aneurinibacillus aneurinilyticus or extract thereof and any combination thereof in an aqueous environment. Such a bath is configured for insertion of a foot or hoof therein such that the aqueous portion of the bath contacts portions of the hoof or foot susceptible to foot rot.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 depicts the results from initial screening showing potential candidates that were used for further screening as described in the Example. The antimicrobial assay shown is against Streptococcus bovis ATCC 700410 (JB1). “A” denotes Aneurinibacillus migulanus and “B” denotes Aneurinibacillus anerinilyticus UNL P2C12 showing inhibition of the growth of Streptococcus bovis. The species identification was performed on the candidate isolates.

FIGS. 2A & 2B depict results of the antimicrobial assay against Streptococcus bovis ATCC 700410 (JB1). FIG. 2A depicts 50 μl of cell extract from 24-hour old culture of Aneurinibacillus migulanus (“A”) and Aneurinibacillus anerinilyticus UNL P2C12 (“B”) showing inhibition of the growth of Streptococcus bovis. FIG. 2B depicts 50 μl of cell free media from 72-hour old culture of Aneurinibacillus migulanus (“A”) and Aneurinibacillus anerinilyticus UNL P2C12 (“B”) showing inhibition of the growth of Streptococcus bovis.

FIGS. 3A & 3B depict results of the antimicrobial assay against Fusobacterium necrophorum ATCC 27852. FIG. 3A depicts 50 μl of cell extract from 24-hour old culture of Aneurinibacillus migulanus (“A”) and Aneurinibacillus anerinilyticus UNL P2C12 (“B”) showing inhibition of the growth of Fusobacterium necrophorum. FIG. 3B depicts 50 μl of cell free media from 72-hour old culture of Aneurinibacillus migulanus (“A”) and Aneurinibacillus anerinilyticus UNL P2C12 (“B”) showing inhibition of the growth of Fusobacterium necrophorum.

FIG. 4 depicts results of the antimicrobial assay control showing that cell growth inhibition is not caused by cell lysis buffer. “A” denotes Streptococcus bovis ATCC 700410 (JB1) and “B” denotes Fusobacterium necrophorum ATCC 27852 in the presence of 50 μl of lysis/extraction buffer. Little to no inhibition is seen compared to FIGS. 2 & 3.

FIG. 5 depicts growth curves for Streptococcus bovis ATCC 700410 (JB1) in the presence of Aneurinibacillus anerinilyticus UNL P2C12 cell extract. The curves demonstrate inhibition of growth of Streptococcus bovis ATCC 700410 (JB1) in the presence of cell extract. The growth curves show that although there is a short lag, the lysis/extraction buffer does not inhibit growth. The culture growth was measured using optical density (OD) at 595 nm. Experiment was done in triplicate and mean values are plotted.

FIG. 6 depicts in vitro induction of acidosis using glucose, showing protection of ruminal pH using the cell extract and live cells. The cell extract shows a dose dependent response. Uninduced—No glucose induction, Induced-Uncontrolled—induced with glucose with no treatment, Cell extract 1000—induced with glucose in the presence of 1000 μl of crude cell extract, Cell extract 500—induced with glucose in the presence of 500 μl of crude cell extract, and Live cells—induced with glucose in the presence of 10⁹ live Aneurinibacillus anerinilyticus UNL P2C12 cells. In the live cells, a rapid initial pH drop is seen due to Aneurinibacillus anerinilyticus UNL P2C12 cells utilizing the glucose added to induce acidosis. However, protection is seen from the live cells 5 hours after induction.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In general, the present disclosure is related to the use of probiotic bacterial strains Aneurinibacillus migulanus and Aneurinibacillus aneurinilyticus, or extracts thereof, for inhibiting the growth of Streptococcus bovis, Fusobacterium necrophorum and Acranobacterium (Actinomyces) pyogenes. Accordingly, the methods of the present disclosure provide treatment for conditions and ailments resulting from overgrowth of Streptococcus bovis, Fusobacterium necrophorum and Acranobacterium (Actinomyces) pyogenes, for example, acidosis, liver abscesses, foot rot, and the like.

“Inhibit” and/or “reduce” or other forms of the words and their synonyms, such as “reducing” or “reduction,” refer to slowing the growth of the so-referenced pathogen and/or lowering its incidence. It is understood that this is typically in relation to some standard or expected value, in other words it is relative, but that it is not always necessary for the standard or relative value to be referred to. For example, “reduces the population of bacteria” in certain instances refer to lowering the amount of bacteria relative to a standard or a control. One preferred control is a similar animal or herd of animals that have not received an administration of the compositions described herein. “Inhibit” and “inhibition” refer to slowing or deterring pathogen growth, including pathogenic bacterial growth that would have otherwise occurred except for the provision of the characterized deterrent.

By “treat” or other forms of the word, such as “treated” or “treatment,” means to administer a composition or to perform a method in order to reduce, prevent, inhibit, break-down, or eliminate a particular characteristic or event or reducing, minimizing and/or eliminating diseases, conditions, ailments and/or symptoms thereof caused by bacterial growth, and in particular, growth of Streptococcus bovis, Fusobacterium necrophorum and Acranobacterium (Actinomyces) pyogenes. In some embodiments, “treatment”, “treating”, or “treat” refer to reversing the progression of conditions or ailments caused by growth of these bacteria. In other embodiments, “treatment”, “treating”, or “treat” refer to slowing or stopping the progression of conditions or ailments caused by growth of these bacteria. This would include slowing or stopping the formation of new abscesses or areas of foot rot or slowing or stopping the growth of existing abscesses or areas of foot rot. In the context of a herd, “treatment”, “treating”, or “treat” can also refer to reducing the number of animals that have liver abscesses or areas of foot rot.

Aneurinibacillus migulanus (formerly known as Bacillus brevis) is a producer of the bacteriocins Carnocyclin A, Zoocin A, Enterolysin A, Linocin M18, and uviB.

Similar properties are seen with Aneurinibacillus aneurinilyticus (formerly known as Bacillus aneurinolyticus), which produces Zoocin A and uviB.

Both Aneurinibacillus migulanus and Aneurinibacillus aneurinilyticus are probiotic bacterial strains isolated from rumen. It has been found that Aneurinibacillus migulanus and Aneurinibacillus aneurinilyticus synthesize and excrete extracts that function as antimicrobial compounds; that is, that inhibit the growth of Streptococcus bovis, Fusobacterium necrophorum and Acranobacterium (Actinomyces) pyogenes, bacterial strains typically linked to acidosis, liver abscesses and foot rot in livestock.

It has been found that by inhibiting Streptococcus bovis, Fusobacterium necrophorum and Acranobacterium (Actinomyces) pyogenes, the methods of the present disclosure can treat diseases, conditions and ailments associated with infection by Streptococcus bovis, Fusobacterium necrophorum and Acranobacterium (Actinomyces) pyogenes. Exemplary conditions include acidosis and/or its direct conditions of liver abscesses and foot rot in livestock.

Other diseases, conditions and ailments can even include sore throats such as observed in human subjects.

The methods of the present disclosure, therefore, are directed to administering Aneurinibacillus migulanus and Aneurinibacillus aneurinilyticus, or extracts thereof, to a subject in need thereof. As used herein, “subject in need thereof” refers to a subset of subjects in need of reducing and/or inhibiting growth of Streptococcus bovis, Fusobacterium necrophorum and Acranobacterium (Actinomyces) pyogenes. In one embodiment, subjects that are in specific need may include livestock, and in particular cattle, horses, pigs, bison, and buffalo, who are susceptible to, or at elevated risk of, infection by Streptococcus bovis, Fusobacterium necrophorum and Acranobacterium (Actinomyces) pyogenes. Subjects may be susceptible to, or at elevated risk of, infection due to environment, feed, and/or lifestyle. While described herein in relation to cattle, it should be understood that other livestock may be administered the bacterial strains and/or extracts thereof without departing from the present disclosure. Further, the bacterial strains or extracts thereof can be administered to subjects other than livestock to inhibit growth of Streptococcus bovis, Fusobacterium necrophorum and Acranobacterium (Actinomyces) pyogenes. For example, the bacterial strains or extracts thereof can be administered to companion animals (e.g., dogs, cats), humans, poultry (e.g., chicken, duck, turkey). Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain conditions and/or ailments.

The term “administering” as used herein includes all means of introducing the bacterial strains and/or their extracts described herein to the subject, including, but not limited to, including, but not limited to, oral (po), intravenous (iv), intramuscular (im), subcutaneous (sc), parenteral, transdermal, inhalation, buccal, ocular, sublingual, vaginal, rectal, and the like. The strains and/or extracts described herein may be administered in unit dosage forms and/or formulations containing conventional nontoxic pharmaceutically-acceptable carriers, adjuvants, and vehicles.

Illustrative formats for oral administration include tablets, capsules, elixirs, syrups, and the like. In some embodiments, oral administration can be in the form of toothpaste such as for the treatment of sore throats in subjects.

In particularly suitable embodiments, the methods of the present disclosure include incorporating Aneurinibacillus migulanus and/or Aneurinibacillus aneurinilyticus or extracts thereof into the diet of the subject as a direct fed microbial product. By way of example, the direct fed microbial product can be used to supplement livestock feed such as cattle feed, horse feed, pig feed, chicken feed, bison feed, buffalo feed, duck feed, turkey feed and the like. Suitable direct fed microbial products can include live cultures or lyophilized preparations.

In another embodiment, the Aneurinibacillus migulanus and/or Aneurinibacillis aneurinilyticus or extracts thereof are administered to the subject as a feed additive such as an additive for use in any one or more of the above livestock feeds. In one particular embodiment, the Aneurinibacillus migulanus and/or Aneurinibacillis aneurinilyticus or extracts thereof are administered in amounts ranging from about 10⁸ to about 10¹¹ colony forming units (CFU). In some preferred forms, the Aneurinibacillus migulanus and/or Aneurinibacillis aneurinilyticus or extracts thereof are administered in amounts ranging from about 10⁹ to about 10¹⁰ CFU, with 10⁹ CFU being particularly preferred.

Illustrative routes for parenteral administration include intravenous, intraarterial, intraperitoneal, epidural, intraurethral, intrasternal, intramuscular and subcutaneous, as well as any other art recognized route of parenteral administration.

Illustratively, administering includes local use, such as when administered locally to the site of inflammation, infection, injury, or defect, or to a particular organ (e.g., liver) or tissue system. Illustrative local administration may be performed during open surgery, or other procedures when the site of inflammation, infection, injury, or defect is accessible. Alternatively, local administration may be performed using parenteral delivery where the bacterial strains/extracts described herein is deposited locally to the site without general distribution to multiple other non-target sites in the subject being treated. It is further appreciated that local administration may be directly in the injury site, or locally in the surrounding tissue. Similar variations regarding local delivery to particular tissue types, such as organs, and the like, are also described herein.

Suitable lubricants include, for example, water-based lubricants, oil-based lubricants, and silicone-based lubricants. Particularly suitable lubricants include, for example, glycerin, hydroxyethyl cellulose, xylitol, carrageenan, and combinations thereof.

In yet other embodiments, administration includes topical administration by use of a topical composition, such as a spray composition, a lotion, an ointment, an aqueous composition, or the like. When used as a topical composition, the composition can include other components typically included in topical sprays, lotions, and/or ointments in combination with the Aneurinibacillus migulanus and/or Aneurinibacillis aneurinilyticus or extracts thereof as known in the topical arts without departing from the present disclosure. In one particularly suitable embodiment, the Aneurinibacillus migulanus and/or Aneurinibacillis aneurinilyticus or extracts thereof are incorporated into a composition in the form of a foot bath such that a subject can contact the foot bath by walking through the bath to apply the composition.

In some embodiments, a therapeutically effective amount of Aneurinibacillus migulanus and/or Aneurinibacillis aneurinilyticus or extracts thereof in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the Aneurinibacillus migulanus and/or Aneurinibacillis aneurinilyticus or extracts thereof can be administered in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

The term “therapeutically effective amount” as used herein, refers to that amount of active compound or agent that elicits the biological or medicinal response in a tissue system, animal or human that is being sought by a researcher, veterinarian, medical doctor or other clinician, which includes alleviation of the symptoms of the disease, condition or ailment being treated. In one aspect, the therapeutically effective amount is that which may treat or alleviate the condition, disease or symptoms of the disease at a reasonable benefit/risk ratio applicable to any medical treatment. However, it is to be understood that the total weekly usage of the Aneurinibacillus migulanus and/or Aneurinibacillis aneurinilyticus or extracts thereof described herein may be decided by the attending physician or veterinarian within the scope of sound medical judgment. The specific therapeutically-effective dose level for any particular patient will depend upon a variety of factors, including the severity of the condition being treated; the specific composition employed; the age, body weight, general health, gender and diet of the patient: the time of administration, route of administration, and the duration of the treatment; drugs used in combination or coincidentally with the Aneurinibacillus migulanus and/or Aneurinibacillis aneurinilyticus or extracts thereof; and like factors well known to the researcher, veterinarian, medical doctor or other clinician of ordinary skill.

It is also appreciated that the therapeutically effective amount is advantageously selected with reference to any toxicity, or other undesirable side effect, that might occur during administration of the Aneurinibacillus migulanus and/or Aneurinibacillis aneurinilyticus or extracts thereof.

In particular embodiments, such as when the Aneurinibacillus migulanus and/or Aneurinibacillis aneurinilyticus or extracts thereof are administered as a feed additive, the Aneurinibacillus migulanus and/or Aneurinibacillis aneurinilyticus or extracts thereof are administered in amounts to provide from about 10⁶ to about 10¹¹ CFU, more preferably 10⁸ to 10¹⁰ CFU, and most preferably about 10⁹ CFU per head per day.

In some embodiments, the Aneurinibacillus migulanus and/or Aneurinibacillis aneurinilyticus or extracts thereof are administered with at least one prebiotic. Prebiotics exert health benefits, which may include, but are not limited to, selective stimulation of the growth and/or activity of one or a limited number of beneficial gut bacteria, stimulation of the growth and/or activity of ingested probiotic microorganisms, selective reduction in gut pathogens, and favorable influence on gut short chain fatty acid profile. Some combinations of Aneurinibacillus migulanus and/or Aneurinibacillis aneurinilyticus or extracts thereof and/or at least one prebiotic will act synergistically with one another. Such prebiotics may be naturally-occurring, synthetic, or developed through the genetic manipulation of organisms and/or plants, whether such new source is now known or developed later. Prebiotics useful in the present disclosure may include soluble starch, yeast extract, oligosaccharides, polysaccharides, and other prebiotics that contain fructose, xylose, soya, galactose, glucose and mannose. Of these, soluble starch and yeast extract are particularly preferred for some forms. Corn has a high amount of soluble starch and distillers grains which comes as a by-product of ethanol fermentation has yeast and yeast extract as by products. Both corn and distillers grains are widely used in cattle diets and as such Aneurinibacillus migulanus and/or Aneurinibacillis aneurinilyticus will grow well when mixed with corn or distillers grains and fed.

More specifically, prebiotics useful in the present disclosure may include soluble starch, yeast extract, polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, and gentio-oligosaccharides.

In an embodiment, the total amount of prebiotics present in the composition may be from about 1.0 g/L to about 10.0 g/L of the composition. More preferably, the total amount of prebiotics present in the nutritional composition may be from about 2.0 g/L and about 8.0 g/L of the composition. In some embodiments, the total amount of prebiotics present in the nutritional composition may be from about 0.1 g/100 kcal to about 1 g/100 kcal. In certain embodiments, the total amount of prebiotics present in the nutritional composition may be from about 0.3 g/100 kcal to about 0.7 g/100 kcal.

The prebiotics of this disclosure can be in the same capsule or formulation as the probiotics, or in a separate dosage form. The prebiotics of this disclosure may also be taken with carbohydrate or fiber to increase their effectiveness.

The compositions of the present disclosure can also include one or more additional active ingredients, excipients, dissolution agents, surfactants, antioxidants, antiseptics, preservatives, penetrants, and combinations thereof.

Various excipients can be mixed with the compositions as would be known to those skilled in the art. Suitable excipients include, for example, microcrystalline cellulose, colloidal silicon dioxide, lactose, starch, sorbitol, cyclodextrin and combinations thereof.

Suitable dissolution agents include, for example, organic acids such as citric acid, fumaric acid, tartaric acid, succinic acid, ascorbic acid, acetic acid, malic acid, glutaric acid and adipic acid, and can be used alone or in combination. These agents can also be combined with salts of the acids, e.g. sodium citrate with citric acid, to produce a buffer system.

Suitable surfactants include, for example, sodium lauryl sulphate, polyethylene separates, polyethylene sorbitan fatty acid esters, polyoxyethylene castor oil derivatives, polyoxyethylene alkyl ethers, benzyl benzoate, cetrimide, cetyl alcohol, docusate sodium, glyceryl monooleate, glyceryl monostearate, glyceryl palmitostearate, lecithin, medium chain triglycerides, monoethanolamine, oleic acid, poloxamers, polyvinyl alcohol and sorbitan fatty acid esters.

Suitable antioxidants include, for example, sodium metabisulfite; tocopherols such as α, β, δ-tocopherol esters and α-tocopherol acetate; ascorbic acid and pharmaceutically acceptable salts thereof; ascorbyl palmitate; alkyl gallates (e.g., propyl gallate, TENOX® PG, TENOX® S-1); sulfites and pharmaceutically acceptable salts thereof butylated hydroxyanisole; butylated hydroxytoluene; and monothioglycerol.

Suitable antiseptics include, for example, chlorhexidine gluconate, glucono delta-lactone, methylparaben, sodium hydroxide, and combinations thereof.

Suitable preservatives include parabens. Suitable parabens include, for example, methylparaben (E number E218), ethylparaben (E214), propylparaben (E216), butylparaben and heptylparaben (E209). Less common parabens include isobutylparaben, isopropylparaben, benzylparaben and their sodium salts.

Suitable penetrants include, for example, sulphoxides (e.g., dimethyl sulphoxide, dimethylacetamide, dimethylformamide), azone (1-dodecylazacycloheptan-2-one or laurocapran), pyrrolidones (e.g., N-methyl-2-pyrolidone), fatty acids (e.g., oleic acid, lauric acid, myristic acid, capric acid), essential oils (e.g., eucalyptus, chenopodium, ylang-ylang, L-menthol), terpenes (e.g., sesquiterpene), terpenoids, oxazolidinones (e.g., 4-decyloxazolidin-2-one), and urea.

In some forms, Aneurinibacillus migulanus and/or Aneurinibacillis aneurinilyticus or extracts thereof are administered separately. In other forms, they are administered together. Preferably the combination of Aneurinibacillus migulanus and/or Aneurinibacillis aneurinilyticus or extracts thereof will act synergistically.

When producing a lyophilized form of Aneurinibacillus migulanus and/or Aneurinibacillis aneurinilyticus, overnight cultures were centrifuged at 3000×g for 15 min at room temperature and the resulting cell pellet was suspended in 10 ml of 20% glycerol in spent media resuspension solution (the media collected after centrifugation was mixed with 50% sterile glycerol to generate a 20% resuspension solution). The resulting cell suspension was snap frozen in liquid nitrogen and was then freeze dried to obtain a freeze dried viable cell product. 10 milligrams of the freeze dried cells were suspended in peptone water and was spread on brain heart infusion agar plates to determine viable colony forming units (CFUs) per milligram of freeze dried product.

The following examples further illustrate specific embodiments of the present disclosure; however, the following illustrative examples should not be interpreted in any way to limit the disclosure.

### EXAMPLES

### Example 1

In this Example, the inhibitory abilities of bacterial strains against Streptococcus bovis and Fusobacterium necrophorum were analyzed.

### Methods

Isolation of the Bacterial Strains

Rumen samples were collected through a rumen fistula from 4 animals. Each animal was on a different diet (animal #6396—deoiled modified distillers plus solubles with added corn oil; animal #6393—Full fat modified distillers plus solubles; animal #6398—dry rolled corn and high moisture corn; and animal #6394—deoiled modified distillers grains plus solubles). Equal amounts (˜10 grams) of each rumen sample were mixed together in a 50 ml conical tube and sterile PBS was added to the top of the tube and shaken vigorously for 5 minutes using a vortexer. Aliquotes of the sample was incubated at 70° C. for 1 hour to reduce the growth of vegetative cells. The sample was serially diluted (10¹-10⁴) and plated on ISP 2, ISP 4 and casein starch agar (Himedia Laboratories, India) with 50 μg/ml cycloheximide (Sigma Aldrich, St Louis, Mo.) and 20 μg/ml Nalidixic acid (Acros Organics, NJ). The plates were incubated anaerobically at 37° C. for 14 days.

Screening for Isolates Against Streptococcus bovis and Fusobacterium necrophorum

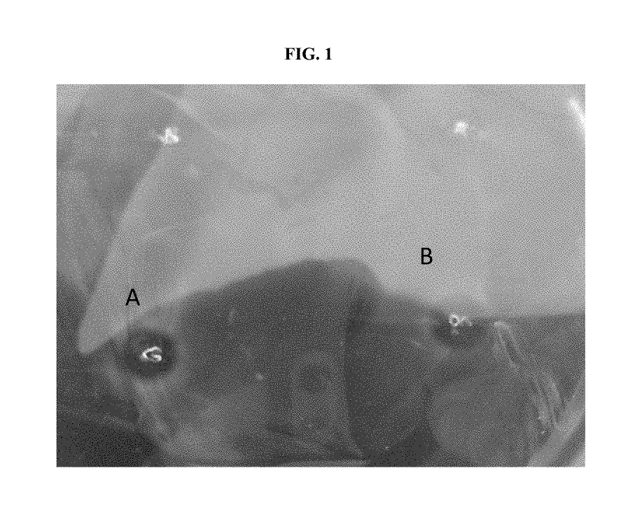

On day 14, colonies were picked into 96-well plates containing tryptic soy broth (TSB) (BD Bacto™, Sparks MD) and were incubated anaerobically and aerobically at 37° C. Screening for isolates that are inhibitory towards Streptococcus bovis and Fusobacterium necrophorum was performed as follows. Briefly, 100 μl of overnight cultures of Streptococcus bovis (ATCC 700410 (JB1)) and 2-day cultures of Fusobacterium necrophorum (ATCC27852), were plated on Brain Heart Infusion (BD BBL™, Sparks, MD) plates and were spotted with isolates. The Streptococcus bovis test organism was grown on TSB and Fusobacterium necrophorum was grown on cooked meat medium (Hardy Diagnostics). The plating was done in duplicate and was incubated both aerobically and anaerobically at 37° C. Plates were checked periodically for clear zones around the isolates, which was indicative of the isolate producing an inhibitory product against the test strain. Positive isolates were regrown from the 96-well plates and glycerol stocks were prepared for archiving, which contained 20% glycerol. Two isolates with the capacity to inhibit the growth of Streptococcus bovis and Fusobacterium necrophorum were identified for further investigation (see FIG. 1).

Characterization of the Microbial Strains

To characterize the microbial isolates and to confirm the identity of the test strains, 16S rRNA gene was sequenced. Briefly, overnight cultures of each strain were used for DNA extraction using the QUICKEXTRACT™—Bacteria DNA extraction kit (Epicenter, Madison, Wis.) according to the manufacturer's protocol. The resulting DNA was used for PCR using universal 16S rRNA primers (27F and 1492R) (Baker et al., Review and re-analysis of domain-specific 16S primers, J. Microbiol. Methods 2003, 55(3): 541-555). The PCR was done using the Terra™ PCR Direct polymerase mix according to the manufacturer's protocol (Clonetech Laboratories, Mountain view, CA). The PCR conditions were, 98° C. for 2 minutes, followed by 35 cycles of 98° C. for 10 seconds, 60° C. for 30 seconds, and 68° C. for 90 seconds. A final extension of 68° C. for 4 minutes was used at the end of the 35 cycles. The PCR products were visualized using agarose gel electrophoresis to ensure a single product. The resulting product was purified using shrimp alkaline phosphatase and exonuclease as described in Sambrook et al., Molecular Cloning: A Laboratory Manual, 2 edn. New York: Cold Spring Harbor Laboratory Press; 1989, and was sequenced from both directions using the 27F and 1492 primers. Sequencing was performed at Eurifins Genomics according to their protocols. The sequencing results generated were matched against the NCBI non-redundant database and the ribosomal database project (Cole et al., The Ribosomal Database Project (RDP-II): sequences and tools for high-throughput rRNA analysis. Nucleic Acids Res 2005, 33:D294-296; Maidak et al., The RDP-II (Ribosomal Database Project). Nucleic Acids Res 2001, 29(1):173-174) to identify the taxonomic classification of the unknown isolates and also to verify the taxonomy of the test strains (SEQ ID NO:1 and SEQ ID NO:2).

Preparing Cell Extracts for Antimicrobial Assays

Based on the 16S sequencing results, both isolates were identified to belong to the genus Aneurinibacillus (A. migulanus (SEQ ID NO:2) and A. anerinilyticus (SEQ ID NO:1)). Based on published studies (Berditsch M, Afonin S, Ulrich A S: The ability of Aneurinibacillus migulanus (Bacillus brevis) to produce the antibiotic gramicidin S is correlated with phenotype variation. Appl Environ Microbiol 2007, 73(20):6620-6628), the cell extracts of the two isolates were purified. Briefly, 5 ml of the cultures were centrifuged at 3000×g for 15 minutes at room temperature. The supernatant was collected and lyophilized to evaluate secreted inhibitory product. Additionally, the cell pellet was resuspended in the lysis buffer (150 mM NaCl and 20 mM HCl) and was incubated at 80° C. for 15 minutes, followed by addition of absolute ethanol (1:1 mix—final concentration of 50%). The slurry was then incubated at room temperature with gentle horizontal agitation for 1 hour. Following, extraction with ethanol, the slurry was centrifuged and the supernatant was collected, which contained the ethanoilic cell extract.

Antimicrobial Assays Against Streptococcus bovis and Fusobacterium necrophorum

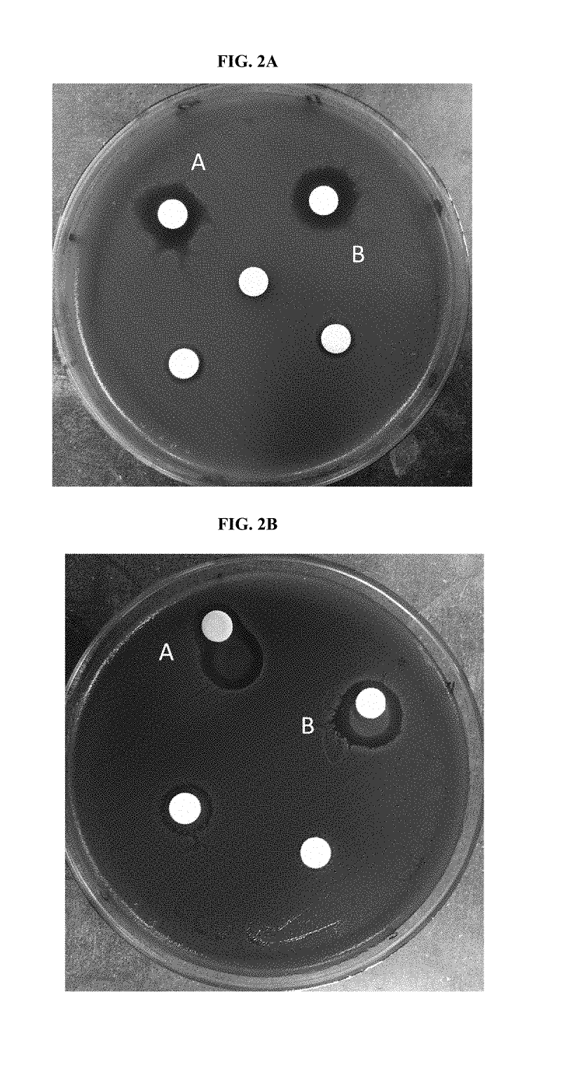

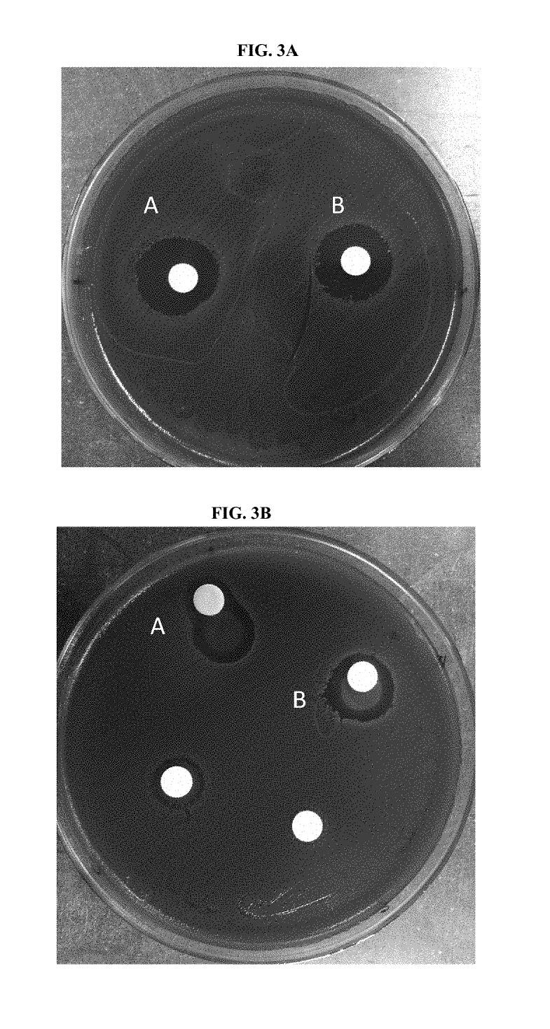

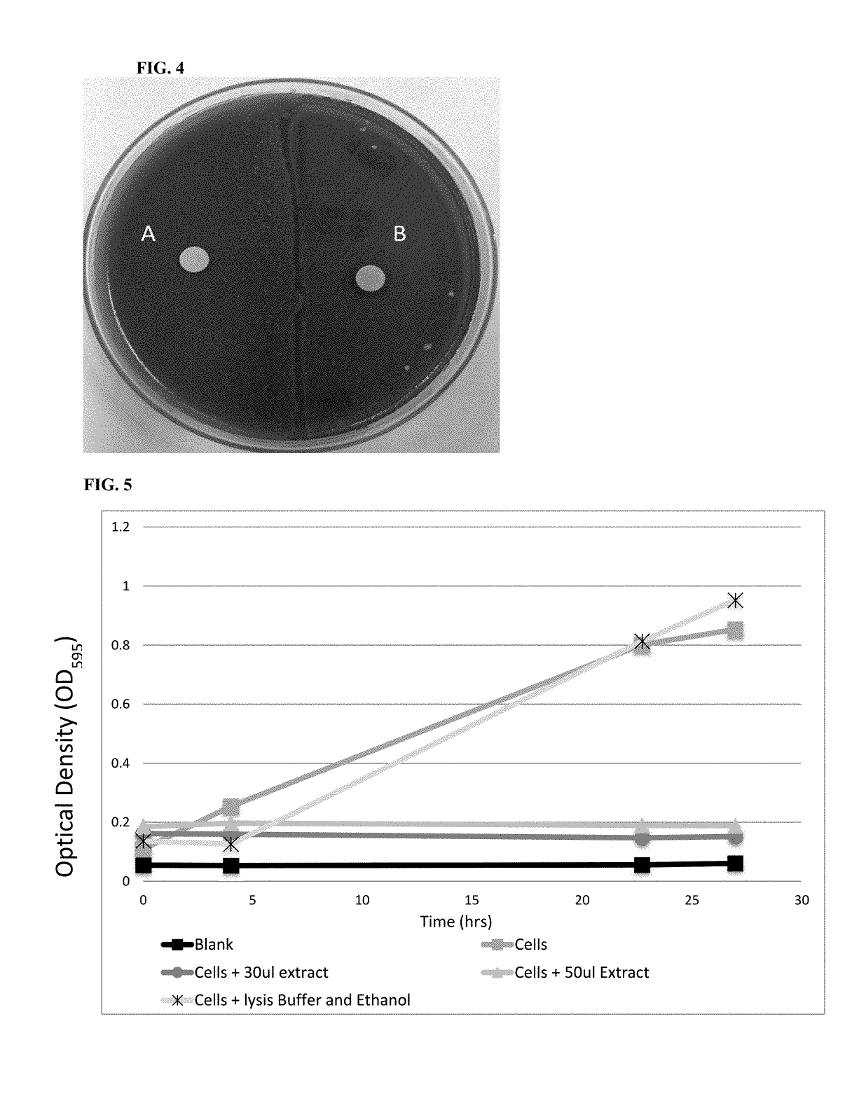

Both the ethanolic cell extract and the lyophilized cell free media were used for agar disk diffusion antimicrobial assays. The Kirby-Bauer Disk Diffusion Susceptibility test was performed as described in Hudzcki: Kirby-Bauer Disk Diffusion Susceptibility Test Protocol: American Society of microbiology; 2009, with the exception of using Mueller Hinton agar plates with 5% sheep blood (purchased from BD BBL™, Sparks, MD) instead of regular Mueller Hinton agar plates without blood. This was done because of the fastidious nature of Fusobacterium necrophorum. In addition to the assays, a control assay was performed to demonstrate that the inhibition of the test strains were a result of the inhibitory compound and not a result of the extraction/lysis buffer used. See FIGS. 2-4 for results of the antimicrobial assays. The blood agar plate assays also demonstrated that the Aneurinibacillus migulanus strain is hemolytic. All incubations were performed in an anaerobic jar at 37° C.

Growth curves of Streptococcus bovis in the presence of the cell lysate of Aneurinibacillus anerinilyticus UNL P2C12

Growth curves for Streptococcus bovis were performed using cell extracts of Aneurinibacillus anerinilyticus UNLP2C12. Briefly, an overnight culture of Streptococcus bovis was diluted in sterile media to bring the OD595 to 0.2 units. One hundred microliters of the diluted culture was dispensed into wells of a 96-well sterile culture plate and the following treatments were imposed each in triplicate. Samples included: Blank (only sterile media), Cells (0.2 OD culture cells), Cells+30 μl of the lysate, Cells+50 μl of the lysate, Cells+lysis buffer. Volumes of all the wells were bought up to 150 μl using sterile media except for the treatment with Cells+50 μl of the lysate as the volume was already 150 μl in this treatment. The plate was incubated anaerobically using an anaerobic jar at 37° C. The cell growth was measured using optical density (OD at 595 nm) at 0, 4, 22.75, and 27 hours of incubation. See Table 1 and FIG. 5. Growth curves for Fusobacterium necrophorum was not performed as this organism is a strict anaerobe and therefore hard to go in and out of the anaerobic jar to maintain growth.

TABLE 1

| | | | | Cells + | Cells + |
|---|---|---|---|---|---|
| | | | Cells + 30 ul | 50 ul | lysis/Extraction |
| Time | Blank | Cells | Extract | Extract | Buffer |
| 0 hours | 0.058 | 0.114 | 0.139 | 0.189 | 0.123 |
| | 0.059 | 0.11 | 0.149 | 0.187 | 0.166 |
| | 0.047 | 0.104 | 0.196 | 0.179 | 0.121 |
| 4 hours | 0.049 | 0.262 | 0.152 | 0.198 | 0.129 |
| | 0.064 | 0.259 | 0.159 | 0.202 | 0.124 |
| | 0.047 | 0.241 | 0.167 | 0.191 | 0.124 |
| 22.75 hours | 0.049 | 0.833 | 0.142 | 0.191 | 0.76 |
| | 0.066 | 0.795 | 0.15 | 0.192 | 0.844 |
| | 0.052 | 0.777 | 0.15 | 0.185 | 0.836 |
| 27.0 hours | 0.047 | 0.814 | 0.137 | 0.186 | 0.764 |
| | 0.071 | 0.828 | 0.15 | 0.195 | 1.137 |
| | 0.064 | 0.917 | 0.168 | 0.184 | 0.957 |

In Vitro Analysis of pH Control During Acidosis Induction

The in vitro analysis was performed. Briefly, Aneurinibacillus anerinilyticus UNL P2C12 was grown overnight and used to inoculate 1 L culture media to grow enough cells for and to extract adequate amounts of cell lysate for the in vitro experiments. The cultures were grown overnight aerobically at 37° C. This was done because the Aneurinibacillus anerinilyticus UNL P2C12 isolate is facultative and can be grown both aerobically and anaerobically, but grows faster aerobically. Part of the overnight growth was flash frozen in liquid nitrogen in the presence of 20% glycerol to use as live culture inoculants in the in vitro experiments. The rest of the culture was used to extract the cell lysate as described above. Glucose was used to induce acidosis as described by Hutton et al. (In Vitro Screening of Plant Resources for Extra-nutritional Attributes in Ruminants: Nuclear and Related Methodologies Springer; 2009: 159-189). The following treatments were used.

| | Rumen Fluid (mL) | D-glucose (g) | Treatment |
|---|---|---|---|
| Uninduced | 10 | 0 | 0 |
| Induced | 10 | 1 | 0 |
| Cell Extract | 10 | 1 | 500 μl |
| Cell Stock | 10 | 1 | 1000 μl |
| Cell Stock | 10 | 1 | 2000 μl |

Briefly, rumen samples were collected anaerobically into a warm thermos as described by Hutton et al. The rumen samples were collected from a fistulated steer 2 hours after feeding. The rumen fluid collected was transported to the laboratory and was opened inside the anaerobic chamber. This process from sampling to opening in the chamber took between 15-20 minutes. Inside the chamber, the rumen fluid was poured to a large beaker and was stirred. Ten milliliters of the rumen fluid was added to each tube inside the anaerobic chamber. All tubes except for the uninduced treatment received 1 gram of glucose and was used to induce acidosis. The treatments were imposed as shown above. The tubes were sealed using butyl rubber stoppers and were crimped with aluminum caps. The gas was equilibrated before bringing the tubes out of the anaerobic chamber using a 23-gauge needle. The sealed tubes were brought out of the anaerobic chamber and were vortexed to mix the contents and glucose and were incubated at 39° C. with gentle agitation (50 rpm).

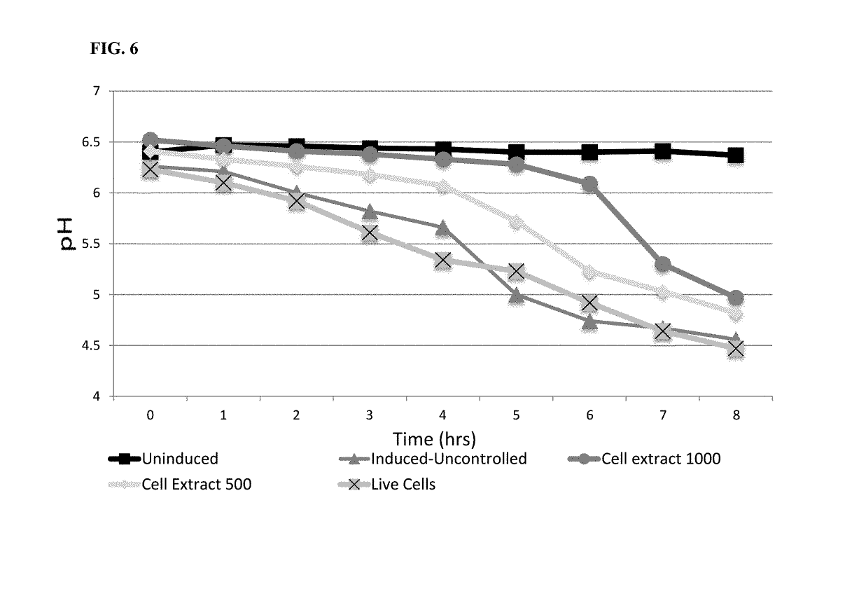

Tubes were removed at 0, 1, 2, 3, 4, 5, 6, 7, 8 hours. Each tube was opened and the pH was recorded. Results of the in vitro assay are shown in FIG. 6.

1. A composition comprising:
a. from about 10⁸ to about 10¹¹ colony forming units (CFU) of a bacterial strain or extract thereof selected from the group selected from the group consisting of Aneurinibacillus migulanus, Aneurinibacillus aneurinilyticus, and any combination thereof; and
b. a prebiotic selected from the group consisting of soluble starch, yeast extract, oligosaccharides, polysaccharides, and other prebiotics that contain fructose, xylose, soya, galactose, glucose, mannose, and any combination thereof in an amount from about 1.0 g/L to about 10.0 g/L of the composition.

2. (canceled)

3. The composition of claim 1, wherein the composition includes both Aneurinibacillus migulanus or an extract thereof, Aneurinibacillus aneurinilyticus or an extract thereof.

4-5. (canceled)

6. The composition of claim 1, wherein the bacterial strain or extract thereof is in a lyophilized form.

7. The composition of claim 6, wherein the lyophilized form further comprises glycerol.

8. The composition of claim 1, wherein the extract of the biological composition comprises at least one bacteroicin selected from the group consisting of Carnocyclin A, Zoocin A, Enterolysin A, Linocin M18, uviB, and any combination thereof.

9. The composition of claim 1, further comprising a component selected from the group consisting of one or more excipients, carriers, active ingredients, dissolution agents, surfactants, antioxidants, antiseptics, preservatives, penetrants, and any combination thereof.

10. A method of inhibiting a bacteria selected from the group consisting of Streptococcus bovis, Fusobacterium necrophorum, Acranobacterium (Actinomyces) pyogenes and any combination thereof in a subject in need thereof, the method comprising administering to the subject an effective amount of a bacterial strain selected from the group consisting of Aneurinibacillus migulanus or an extract thereof, Aneurinibacillus aneurinilyticus or an extract thereof and any combination thereof.

11. The method as set forth in claim 10, wherein the bacterial strain or extract thereof is administered in a form selected from the group consisting of a direct fed microbial product, a feed additive, a toothpaste, a topical composition, or any combination thereof.

12. The method as set forth in claim 11, wherein the direct fed microbial product can be used to supplement at least one livestock feed selected from the group consisting of cattle feed, pig feed, chicken feed, bison feed, buffalo feed, duck feed and turkey feed.

13-14. (canceled)

15. The method as set forth in claim 11, wherein the topical composition is in a form selected from the group consisting of a spray composition, a lotion, an ointment, a footbath, or any combination thereof.

16-17. (canceled)

18. The method as set forth in claim 10, wherein the subject is selected from the group consisting of livestock, companion animal, and poultry.

19. The method as set forth in claim 10, further comprising the step of administering an effective amount of a prebiotic.

20. The method as set forth in claim 10, wherein the effective amount of the bacterial strain comprises from about 10⁸ to about 10¹¹ colony forming units (CFU).

21-43. (canceled)

44. A method of reducing the incidence or severity of a condition selected from the group consisting of acidosis, liver abscesses, foot rot, or any combination thereof in a subject in need thereof, the method comprising administering to the subject an effective amount of a bacterial strain selected from the group consisting of Aneurinibacillus migulanus or an extract thereof, Aneurinibacillus aneurinilyticus or an extract thereof, and any combination thereof.

45. The method as set forth in claim 44, wherein the bacterial strain or extract thereof is administered in a form selected from the group consisting of a direct fed microbial product, a feed additive, a topical composition, a toothpaste, or any combination thereof.

46. The method as set forth in claim 45, wherein the direct fed microbial product can be used to supplement at least one livestock feed selected from the group consisting of cattle feed, pig feed, chicken feed, bison feed, buffalo feed, duck feed and turkey feed.

47. (canceled)

48. The method as set forth in claim 44, wherein the effective amount of the bacterial strain administered is from about 10⁶ to about 10¹¹ cells per head per day.

49. The method as set forth in claim 44, wherein the subject is selected from the group consisting of livestock, companion animal, and poultry.

50. The method as set forth in claim 44, further comprising the step of administering an effective amount of a prebiotic.

51. The method as set forth in claim 44, wherein the effective amount of the bacterial strain comprises from about 10⁸ to about 10¹¹ colony forming units (CFU).

52-68. (canceled)


SEQUENCE LISTING (2 sequences)

SEQ ID NO: 1 - DNA, 804 bp, Aneurinibacillus aneurinolyticus
cttgctcttcggcggttagcggcggacgggtgagtaacacgtaggcaacctgcctgtacgactgggataactccgggaaaccggagctaataccggatacgtttttcagaccgcatggtctgaaagagaaagacctttggtcacgtacagatgggcctgcggcgcattagctagttggtggggtaacggcctaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgatgaaggttttcggatcgtaaagttctgttgttagggaagaaccgccgggatgacctcccggtctgacggtacctaacgagaaagccccggctaactacgtgccagcagccgcggtaatacgtagggggcaagcgttgtccggaattattgggcgtaaagcgcgcgcaggcggcttcttaagtcaggtgtgaaagcccacggctcaaccgtggagggccacttgaaactgggaggcttgagtgcaggagaggagagcggaattccacgtgtagcggtgaaatgcgtagagatgtggaggaacacccgtggcgaaggcggctctctggcctgtaactgacgctgaggcgcgaaagcgtggggagcgaacaggattagataccctggtagtccacgccgtaaacgttgagtgctaggtgttggggactccaatcctcagtgccccagctaacgcaataagcactccgcctggg

SEQ ID NO: 2 - DNA, 755 bp, Aneurinibacillus migulanus
cagagcttgctcttcggcggttagcggcggacgggtgagtaacacgtaggcaacctgcctgtacgactgggataactccgggaaaccggagctaataccggatacttctttcagaccgcatggtctgaaagggaaagacctttggtcacgtacagatgggcctgcggcgcattagctagttggtggggtaacggcctaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgatgaaggttttcggatcgtaaagttctgttgttagggaagaaccgccgggatgacctcccggtctgacggtacctaacgagaaagccccggctaactacgtgccagcagccgcggtaatacgtagggggcaagcgttgtccggaattattgggcgtaaagcgcgcgcaggcggcttcttaagtcaggtgtgaaagcccacggctcaaccgtggagggccacttgaaactgggaagcttgagtgcaggagaggagagcggaattccacgtgtagcggtgaaatgcgtagagatgtggaggaacacccgtggcgaaggcggctctctggcctgtaactgacgctgaggcgcgaaagcgtggggagcgaacaggattagataccctggtagtccacgccgtaaacgttgagtgctaggtg